(19) United States
(12) Patent Application Publication
(10) Pub. No.: US 2010/0191183 A1
(43) Pub. Date: Jul. 29, 2010

(54) Tip for an Insertion Device

(75) Inventors: Allan Tanghoej, Kokkedal (DK); Pernille Merethe Guldager, Lyngby (DK); Jokum Christian Voss, Espergaerde (DK)

Correspondence Address: COLOPLAST A/S;Attention: Corporate Patents, Holtedam 1, DK-3050 Humlebaek, DK

(73) Assignee: COLOPLAST A/S, Humlebaek (DK)

(21) Appl. No.: 12/668,884

(22) PCT Filed: Jul. 14, 2008
(86) PCT No.: PCT/DK2008/050178

§ 371 Date: Mar. 29, 2010

(30) Foreign Application Priority Data
Jul. 13, 2007 (DK) PA 2007 01040

(51) Int. Cl. A61M 29/00

(52) U.S. Cl. 604/96.01

(57) ABSTRACT

An insertion device for introduction into an opening in the body includes a tip section, a balloon section and a handling section. The balloon section has an expandable balloon-element and an inner balloon part, where the tip section, the inner balloon part and the handling section each have one or more liquid channels) in communication with each other. The inner balloon part and the handling section further have an air channel in communication with each other with an outlet in the inner balloon part. The balloon-element is radially expandable relative to the longitudinal axis of the insertion device by air/fluid injection through the air channel, and the tip section and the balloon-element are one integral element.

The invention relates to an insertion device including an inflatable balloon-element where the tip of the insertion device and the balloon-element are one integral element. The tip is soft and flexible. The insertion device includes a handling part, which is more rigid.

### BACKGROUND

Foley-type catheters are tube-like devices that are used to drain urine from a user's bladder. Foley catheters are inserted through the urethra and are typically held in place with an inflatable balloon. The balloon is in a deflated position when the catheter is inserted at first. Once the catheter is in the proper position, the balloon is inflated with a fluid. The inflated balloon is larger in diameter than the diameter of the urethra and thereby physically inhibits movement of the catheter. Foley catheters are also known as “indwelling” catheters because they are designed to be left in place for a period of time. Other types of catheters or insertion devices also comprise a tube-like element and a balloon disposed on the outside surface of the catheter. An example of this is rectal catheters, which are typically used in connection with anal irrigation. Anal irrigation is often used to stimulate the peristaltic function of the intestines and thus reduce constipation. Especially paralysed persons, e.g. suffering from spinal cord injuries, spinal bifida or multiple sclerosis suffer from decreased peristaltic function and thereby reduced function of the bowel system. In connection with anal irrigation, irrigation liquid (typically water) flows into the rectum through a rectal catheter, which is held in place in the rectum by an inflated balloon. Likewise, tube-like devices having a balloon disposed on the outside surface are used for tracheotomy devices, catheters and other types of insertion devices.

U.S. Pat. No. 3,902,492 provides a catheter for irrigation of a body cavity. The catheter has a drainage tube for the removal of fluid from the bladder. It is also provided with a second axial bore in its wall, which terminates in an opening beneath a sheath of expandable material, which can be inflated by fluid applied under pressure through the second axial bore, to secure the catheter in place.

Such a catheter is typically made of flexible plastics, such as PVC and the sheath is made as a separate element of rubber latex or other expandable material.

There is still a need for an improvement of insertion devices of the type mentioned above.

### SUMMARY OF THE INVENTION

The invention relates to providing a tip and a balloon-element in one integral element, meaning that there is no connection at the transition between the tip and the balloon element. Thereby, the surface of the insertion device will be smooth, in a way that stress and maceration of the internal surface of the insertion channel is avoided. The integral element is made of a material having a rather low Shore-value—e.g. silicone—such that the tip is soft. The integral element may be made by injection-moulding. The balloon-section of the insertion device has two parts—a balloon-element and an inner balloon part.

### DETAILED DESCRIPTION OF THE INVENTION

In a first aspect the invention relates to an insertion device for introduction into an opening in the body, comprising a tip section, a balloon section and a handling section, the balloon section comprising an expandable balloon-element and an inner balloon part, where the tip section, the inner balloon part and the handling section each comprise one or more liquid channel(s) in communication with each other and where the inner balloon part and the handling section further comprise an air channel in communication with each other, with an outlet in the inner balloon part and where the balloon-element is radially expandable relative to the longitudinal axis of the insertion device by air/fluid injection through the air channel, wherein the tip section and the balloon-element are one integral element.

The tip section and the balloon-element as an integral element makes it possible to avoid ridges and grooves due to a connection at the transition between the tip section and the balloon-element. Thereby, the surface of the insertion device can be made smooth, in a way that injury of the tissue in the body opening upon insertion of the insertion device is reduced or completely avoided.

Furthermore, such a balloon-element and tip section may be injection-moulded, meaning that all parts of the insertion device may be injection-moulded. This is a cheap and easy way of providing an insertion device.

A catheter according to this invention may be used as a Foley catheter. It may also be used in connection with anal irrigation systems. Another example of use is in connection with tracheotomy. A Foley catheter may be used as an indwelling catheter, where the balloon-element is used to hold the catheter in place inside the urethra, in a way that urine may flow out through the catheter and into a collection bag. In connection with anal irrigation systems, a rectal catheter is inserted into the rectum, the balloon-element is inflated to maintain the rectal catheter in position and then irrigation liquid flows through the catheter and into the rectum. In connection with tracheotomy, a tracheotomy catheter is inserted through a hole in the trachea (the windpipe) to blow air into the lungs of a patient. To maintain the catheter in position in the trachea, a balloon-element may be provided at the catheter. Insertion devices according to the invention may also be used for this purpose.

The insertion device comprises a proximal end and a distal end. It should be understood herein that by referring to the proximal end of an element or a part, reference is made to a side or a section of an element or a part, which is closest to the user (e.g. the person to whom the anal irrigation or emptying of the bladder is performed) right before the insertion device is inserted into the body opening (e.g. the rectum or the urethra). Likewise, the distal end is the end opposite the proximal end.

The insertion device according to the invention also comprises one or more liquid channel(s) extending through the insertion device from the distal end to the proximal end (or to eyelets near the proximal end). For a rectal catheter the liquid channel(s) are used for the irrigation liquid. For an indwelling catheter the liquid channel(s) are used for the urine. The insertion device further comprises an air channel extending from the distal end through the handling section and partly through the inner balloon part. The air channel has one or more outlets in the inner balloon part. These outlets are disposed under the balloon-element, in a way that the balloon-element is inflated by blowing air or fluid (e.g. water) through the air channel from the distal end of the insertion device to the outlets. Typically, the air or fluid would also be evacuated through the air channel.

The material for the integral element is selected to be biocompatible. This means that many types of material may be used—for example silicone, PVC (Poly Vinylchloride), Chloroprene, TPU (Thermo Plastic Urethane), TPE (Thermoplastic Elastomer), Isoprene etc. To further be environmentally friendly the following materials are preferred: Silicone TPE (Thermoplastic Elastomer) and Isoprene. The last two types of material have a higher compression set and for this reason silicone may be preferred. Silicone has a very low compression set and has almost ideal elastic material properties.

The Shore-value and the rigidity of the insertion device should be balanced between flexible and soft (in order to prevent damage or injury to the tissue) and on the other hand easy to insert (requiring rigidity). The Shore-value of the integral element is preferably between 30 and 50 Shore A.

The handling section may also be known as the handling part as it may be a part separate from the other parts constituting the insertion device.

The handling section is relatively rigid, in a way that the user is able to control the movement of the insertion device at least partly. The handling section may be made of PVC (Polyvinyl Chloride, e.g. polyone 8181 Blue Medical Grade Plastisol), SI (Silicone), PE (Polyethylene), PP (Polypropylene), TPE (Thermoplastic Elastomer), TPU (Thermoplastic Urethane), synthetic rubbers such as Isoprene and chloroprene.

The handling section may further comprise a plug, which may be formed as a separate element of the same kind of material as the handling section, but may also be made as another material. It may be an integrated element.

The handling section comprises a through-going liquid channel forming part of the liquid channel of the insertion device. The handling section further comprises a through-going air channel forming part of the air channel of the insertion device.

The shore-value of the handling section may be between 60 and 90 Shore A.

The balloon section comprises at least two parts—an inner balloon part and a balloon-element. The balloon-element is expandable under influence of an internal air or fluid pressure. The balloon-element is integral with the tip section, in a way that the balloon-element and the tip section are one element. In other words the soft tip has a balloon-element. The balloon-element may be filled with either air or fluid (e.g. water), whereby the balloon-element expands, in a way that its radial extent is considerably larger than other parts or sections of the catheter. It may be made of a relatively thin material such that it is stretchable. When silicone is used it may be approximately 0.35 mm, which provides for a stretchability enabling the balloon to stretch from a diameter of approximately 1 cm to approximately. 7 cm. The balloon-element may also be folded such that expansion occurs as a result of the folds unfolding themselves. When a folded balloon-element is used, the balloon-element may be less stretchable.

As with other sections of the insertion device, the inner balloon part comprises a through-going liquid channel in the longitudinal direction. The inner balloon part further comprises an air channel extending partly through the inner balloon part. The liquid channel and the air channel both form parts of the liquid channel and the air channel of the insertion device.

The inner balloon part may be made as a tube-like element having largely the same rigidity as the handling part. In another embodiment, the inner balloon part may be more flexible than the handling section when subjected to bending. A more flexible balloon section would enable the insertion device to bend slightly if a curve in the body opening is met. The flexibility may be obtained in at least three different ways. One way is to make the inner balloon part by the same type of material as the handling part and then weaken it by providing it with transverse ribs. The thickness, depth and number of ribs may be adjusted in relation to the material constituting the inner balloon part and the required flexibility. Another way of imparting an increased flexibility to the inner balloon part is by making a tube-like core-element including the liquid channel of a flexible material, where the material has lower modulus of elasticity than the material of the handling section. The flexible core-element may subsequently be braced slightly in the longitudinal direction by providing it with longitudinal ribs of a more rigid material. A flexible inner balloon part may also be obtained by providing the inner balloon part as a tube-like element of a material having the proper flexibility.

When the inner balloon part comprises transverse ribs there is a risk that the air may be entrapped in the balloon as the balloon is evacuated. This is due to the balloon-element collapsing on top of the ribs, thereby preventing the air proximal of the collapsed balloon-element from evacuating through the outlets. Due to the ribs air would still be present, proximate of the collapsed balloon-element and the ribs would block the passage of air along the longitudinal direction of the inner balloon part—that is transversely of the ribs. To avoid this, the inner balloon part may comprise a longitudinal extending groove across all of the ribs. Thereby, the air would always be able to evacuate from the end near the tip section (the proximal end) to the end near the handling section (the distal end), where the outlet from the air channel is located. From there the air would escape through the air channel of the handling section and out. The longitudinal groove may be extending in the longitudinal direction or it may be spiraling along the length.

If the inner balloon part is a tube-like element or provided with longitudinal grooves the air would be able to evacuate unhindered in the longitudinal direction.

A snap-fit connection between inner balloon part and handling section is preferred. The inner balloon part and the handling section may also be glued or welded together. Furthermore, the handling section and the inner balloon part may be a single integral part.

The balloon-element is preferably attached by a snap fit or friction fit connection to the inner balloon part—that is at the distal end of the balloon-element. The balloon-element may also be welded or glued to the inner balloon part. It is important that the balloon-element does not separate from the inner balloon part at the distal end of the balloon-element.

The length of the inner balloon part may correspond to the length of the balloon-element, plus the connecting parts at proximal and distal end of the inner balloon part. This would typically be the case when the insertion device was used as a rectal catheter. If the insertion device were to be used as an indwelling catheter, the inner balloon part would preferably be disposed as a flexible tube-like element with a length significantly longer than the balloon-element e.g. 10 to 20 times longer.

In an embodiment, the handling section and the inner balloon part comprise a first material and the integral element comprises a second material. In a related embodiment, the first material comprises Polyethylene and the second material comprises Silicone.

The tip section and the balloon-element may be injection-moulded or moulded by a rubber moulding. The tip section may include an O-ring in the transition between the tip section and the balloon-element for snap-attachment to the inner balloon part. In that case the tip section and the balloon-element may be insert-moulded or two-component moulded. Snap-fitting the tip section to the inner balloon part allows a control over the strength of the connection. This provides the possibility of separating the tip section from the inner balloon part when the pressure inside the balloon gets too high—in other words, an excessive pressure would release the tip section from the inner balloon part. The balloon-element and tip section would still be connected to the inner balloon part and handling section in the end opposite the tip-section. In other words, the integral element does not separate completely from the remaining parts of the insertion device. When the tip separates from the inner balloon part, the air entrapped in the balloon will be able to exit the insertion device through the tip and enter into the rectum, urethra or other body openings. This prevents the balloon-element from bursting inside the body of the user or patient. The Example relates to testing of the separation effect.

The tip section may be closed in the end (in the proximal end) as urinary of rectal catheters usually are. In this embodiment, the tip section may be provided with one or more eyelet(s) at the side of the tip section. In another embodiment, the tip section may be open in the end, which is usually the case in connection with tracheotomy devices.

The tip section may be rounded in the end allowing for easy insertion into the rectum or urethra.

### BRIEF DESCRIPTION OF THE DRAWING

FIG. 1 illustrates a rectal catheter with a balloon for fixation,

FIGS. 2a-2c illustrate the three parts or elements of the rectal catheter such that FIG. 2a illustrates the integral element (the tip section and the balloon-element), FIG. 2b illustrates the handling section and FIG. 2c illustrates the inner balloon part,

FIG. 3 illustrates a cross-sectional view of the rectal catheter,

FIGS. 4a-4c illustrate cross-section views of the three elements of the rectal catheter corresponding to FIGS. 2a-2c and

FIG. 5 illustrates an indwelling catheter according to the invention.

### DETAILED DESCRIPTION OF THE DRAWING

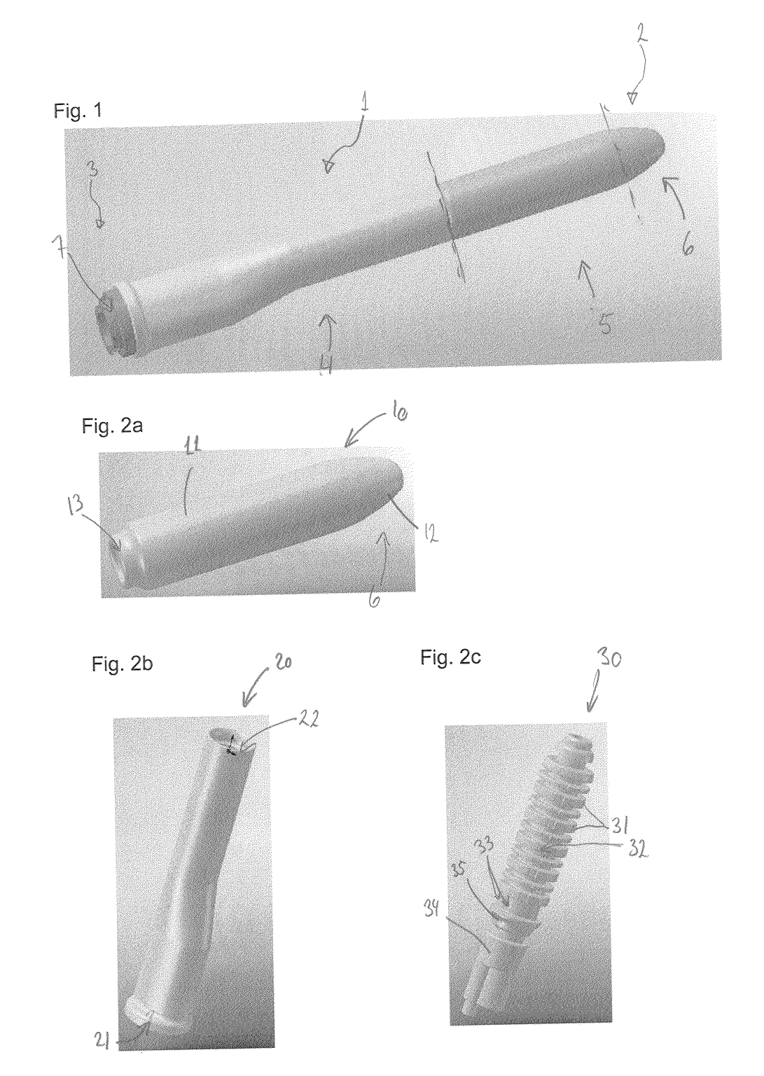

FIGS. 1 to 4 illustrate one embodiment of an insertion device 1 according to the invention. In FIG. 1, the insertion device in the form of a rectal catheter is illustrated. The rectal catheter 1 has two ends—an insertion end 2 corresponding to a proximal end comprising a tip and a connecting end 3 for connection to a drainage tube or a fluid providing tube or tubes (not shown). The connecting end may also be known as the distal end. In the longitudinal direction, the insertion device comprises three sections, a handling section 4 near the connecting end, a balloon section 5 and a tip section 6 near the insertion end. Preferably, all sections have cylindrically shaped bodies, however, oval cross-sections are also conceivable. When assembled, the three sections may taper slightly such that the inserting end 2 has a smaller cross-section than the connecting end 3. At the distal end, the catheter 1 is provided with a double Luer-lock connector 7 for attaching the catheter 1 to tubes providing the irrigation liquid and the air or fluid for inflating the balloon-element.

FIG. 2a illustrates the integral element 10, which forms the balloon-element 11 and the tip section 6. The tip section 6 comprises a tip part 12. The integral element 10 further comprises an O-ring 13 at the distal end for snap-fitting to the inner balloon part (see FIG. 2c).

FIG. 2b illustrates the handling part 20 of the handling section. The handling part 20 comprises a distal end having a female part 21 of a Luer-connection adapted for connection with the Luer-lock connector 7 (see FIG. 1). The proximal end of the handling part 20 has a female part 22 of a friction-fit connection adapted to cooperate with a male part of the inner balloon part (see FIG. 2c). The handling part 20 is typically made of a rather rigid material, as a user needs to be able to steer and control the rectal catheter 1 by holding the handling part 20.

FIG. 2c illustrates the inner balloon part 30. In this embodiment, the inner balloon part 30 comprises ribs 31 for imparting increased flexibility on the inner balloon part. To avoid entrapping of air at the proximal end of the inner balloon part 30 the ribs 31 are provided with a longitudinal extending groove 32. Two outlets 33 from the air channel are provided. The inner balloon part 30 also comprises a male part 34 of a friction-fit connection for connecting the inner balloon part 30 with the handling part 20 (see FIG. 2b). Furthermore, the inner balloon part 30 comprises a snap-fitting part 35 for snap-fitting with the distal O-ring 13 of the integral element 10 (see FIG. 2a). The snap-fitting part 35 is in the form of an upstanding rib tapering towards the distal end. In this embodiment, the outer circumference of the upstanding rib is slightly larger than the circumference of the most distal end of the balloon-element—the O-ring 13. This provides for a secure snap-fit between the two elements.

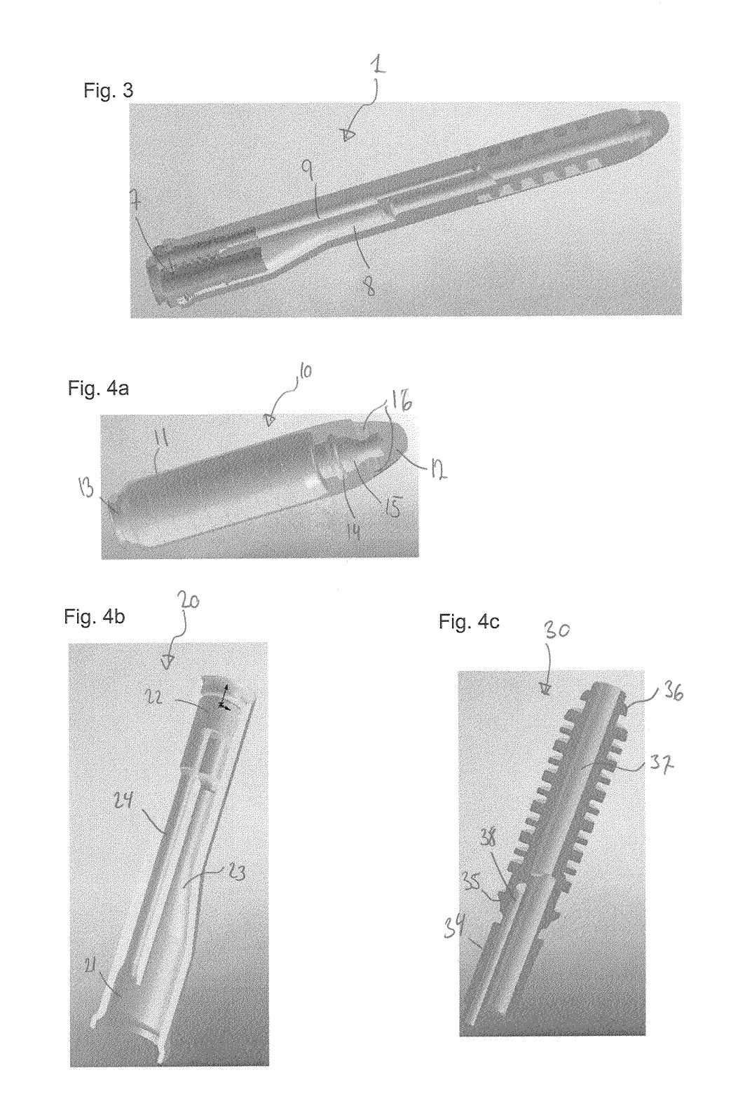

FIG. 3 illustrates a cross-sectional view of the insertion device 1. The through-going liquid channel 8 and the air-channel 9 are shown. Furthermore, the connector 7 may also be seen.

FIG. 4a illustrates a cross-sectional view of the integral element 10 showing the balloon-element 11, tip part 12 and the distal O-ring 13 for snap-fitting with the inner balloon part. The integral element 10 further has a proximal O-ring 14 disposed at the transition between the tip part 12 and the balloon-element 11. This proximal O-ring 14 is adapted for snap-fitting to the proximal end of the inner balloon part (see FIG. 4c). The integrated proximal O-ring 14 may be provided of the same material as the remaining part of the integral element or of a harder material. The tip part 12 comprises the proximal part 15 of the liquid channel 8 of the catheter 1 terminating in two eyelets 16.

FIG. 4b illustrates a cross-sectional view of the handling part 20 comprising the female part 21 of a Luer-lock connection at the distal end of the handling part 20 and a female part 22 of a friction-fit connection at the proximal end. The handling part 20 further comprises the distal parts of the liquid channel 23 and the air channel 24. They are both through-going through the handling part 20.

FIG. 4c illustrates a cross-sectional view of the inner balloon part 30 comprising the male part 34 of the friction-fit connection adapted for cooperating with the female part 22 at the handling part 20 (see FIG. 4b). The inner balloon part 30 also comprises the snap-fitting part 35 adapted for cooperation with the O-ring 13 at the distal end of the integral element 10. At the proximal end of the inner balloon part 30, another snap-fitting part 36 in the form of an upstanding rib is provided. The upstanding rib tapers towards the proximal end. The proximal snap-fitting part 36 is adapted for cooperation with the proximal O-ring 14 of the integral element 10. The snap-fit between the snap-fitting part 36 and the O-ring 14 provides for a connection, which separates under influence of an excessive pressure inside the balloon-element 11. Thereby, the tip part 12 would detach from the inner balloon part 30 and air entrapped in the balloon-element 11 would be able to flow through the liquid channel 15 of the tip part 12 and into the rectum through the eyelets 16. This prevents the balloon-element 11 from bursting inside the rectum. The inner balloon part 30 further comprises a part 37 of the liquid channel of the catheter and the proximal part 38 of the air channel. The air channel terminates in outlets 33 provided in the inner balloon part (see FIG. 2c).

The rectal insertion device 1 illustrated in FIGS. 1 to 4 is between 100 and 150 mm long—more precisely approximately 115 mm long. The integral element 10 constituting the tip section and the balloon-element is approximately 50 mm long. The tip part 12 is between 12 and 16 mm thick and approximately 15 mm long. The balloon-element 11 is approximately 35 mm long and 0.35 mm thick. The eyelets 16 are placed approximately 8 mm distally from the proximal part of the tip part 12.

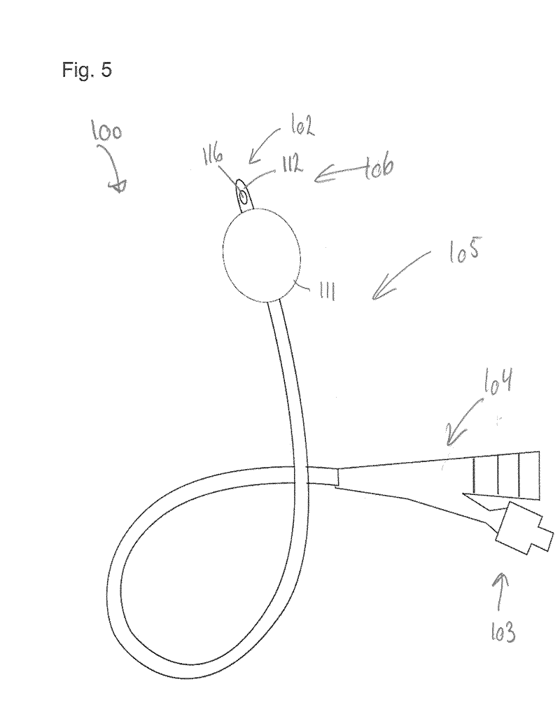

FIG. 5 illustrates a urinary drainage device—a Foley type catheter or an indwelling catheter 100. The device comprises a catheter of approximately 420 mm in length. The indwelling catheter 100 comprises a proximal end 102 and a distal end 103 and a connector 107 at the distal end 103. The indwelling catheter is divided in three sections, a handling section 104, a balloon section 105 and a tip section 106 including the actual tip part 112. The balloon section 105 includes the balloon element 111. The tip part 112 includes the eyelets 116 into the catheter 100. The length of the tip part 112 and the length of the balloon section is approximately 20 mm each such that the overall length of the integral element is approx. 40 mm. The liquid channels protrude at eyelets 116 approximately 12 mm from the proximal end of the tip section corresponding to the proximal end 102 of the catheter. The balloon-element 111 is inflated for illustration purposes.

### Example

### Test of Separation

This example relates to testing of separation of the tip-section from the inner balloon part. Six different types of prototypes were used using three different handling sections combined with two different integral elements constituting the balloon-elements and the tip sections. The handling sections and the inner balloon part were all made of PE. The integral elements were made of silicone with a shore-value of approx. 30 Shore A. The elements included a ring-element (an O-ring) adapted for snap-fitting with the inner balloon part. The ring-element of element no. 1 was made of the same material as the remaining part of the integral element. The ring-element of element no. 2 was made of a harder material with a shore-value of approximately 40 Shore A. For all prototypes the integral element was snap-fitted to the inner balloon part and to a connection between the handling section and the inner balloon part. Furthermore, a plug in form of a Luer-lock connector was attached to the end of the handling section allowing for connection to a water tube. For each type of handling section, 10 insertion devices were produced, 6 with integral element no. 1 and 4 with integral element no. 2. Test-results were obtained for 13 insertion devices. The remaining insertion devices were either not tested or were not fitted probably to the test rig, such that the results were untrustworthy.

The test was made by inflating the balloon-element using water and the test was performed while the insertion device was immersed in water. Thereby, equal pressure was initially obtained on the inside and the outside of the balloon. The table below illustrates the results.

| Handling | | | Peak | |
|---|---|---|---|---|
| section type | Sample no. | Inlet pressure | pressure | Element no. |
| Borealis M8200 | 1 | 65 | 123 | no. 1 |
| | 2 | 63 | 133 | |
| | 3 | 58 | 138 | no. 2 |
| | 4 | 61 | 134 | |
| Meliflex M8002 | 1 | 70 | 135 | no. 1 |
| | 2 | 69 | 122 | |
| | 3 | 58 | 122 | |
| | 4 | 63 | 135 | no. 2 |
| Meliflex 2605A | 1 | 83 | 129 | no. 1 |
| | 2 | 68 | 129 | |
| | 3 | 70 | 127 | |
| | 4 | 62 | 137 | no. 2 |
| | 5 | 60 | 145 | |

All pressures were measured in hPa.

The tests using integral element no. 1 showed that at a pressure of approximately 120-135 hPa would separate the tip section from the inner balloon part, in a way that the balloon deflated. The tests using integral element no. 2 showed that at a pressure of approximately 130-145 hPa the tip section was separated from the inner balloon part. In none of the 13 tests did the balloon burst.

1. An insertion device for introduction into an opening in the body comprising a tip section, a balloon section and a handling section, the balloon section comprising an expandable balloon-element and an inner balloon part, where the tip section, the inner balloon part and the handling section each comprise one or more liquid channel(s) in communication with each other and where the inner balloon part and the handling section further comprise an air channel in communication with each other with an outlet in the inner balloon part and where the balloon-element is radially expandable relative to the longitudinal axis of the insertion device by air/fluid injection through the air channel, wherein the tip section and the balloon-element are one integral element.

2. An insertion device according to claim 1, wherein the handling section and the inner balloon part comprise a first material and the integral element comprises a second material.

3. An insertion device according to claim 2, wherein the first material comprises Polyethylene and the second material comprises Silicone.

4. An insertion device according to claim 1, wherein the integral element the tip section and the balloon-element is made of a material with a Shore-value between 30 and 50 Shore A.

5. At insertion device according to claim 1, wherein the handling section is made of a material with a Shore-value of between 60 and 90 Shore A.

6. An insertion device according to claim 1, wherein the inner balloon part is more flexible than the handling section when subjected to a bending force.

7. An insertion device according to claim 6, wherein the inner balloon part comprises transverse ribs.

8. An insertion device according to claim 7, wherein the inner balloon part comprises a longitudinal extending groove across an of the ribs.

9. An insertion device according to claim 6, wherein the inner balloon part comprises a flexible core-element including the liquid channel and longitudinal extending ribs placed along the core.

10. An insertion device according to claim 1, wherein the tip section is attached in a way that an excessive pressure releases the tip section from the inner balloon part.

11. An insertion device according to claim 1, wherein the integral element comprising the tip section and the balloon-element is attached to the inner balloon part at the distal end of the balloon-element.

12. An insertion device according to claim 1, wherein the handling section and the inner balloon part are one integral element.

13. An insertion device according to claim 1, wherein the handling section and the inner balloon part are two separate elements that are snap-fitted together.

14. An insertion device according to claim 1, wherein the insertion device is a rectal catheter.
